(19) United States
(12) Patent Application Publication
(10) Pub. No.: US 2005/0177221 A1
(43) Pub. Date: Aug. 11, 2005

(54) Ostial stent

(76) Inventor: Jihad A. Mustapha, Ada, MI (US)

Correspondence Address: WARNER NORCROSS & JUDD LLP, 900 FIFTH THIRD CENTER, 111 LYON STREET, N.W., GRAND RAPIDS, MI, 49503-2487, US

(21) Appl. No.: 10/773,709

(22) Filed: Feb. 6, 2004

(51) Int. Cl. A61F 2/06

(52) U.S. Cl. 623/1.11

(57) ABSTRACT

The specification discloses a cardiovascular ostial stents, a ostial stent balloon, and methods for their use. The stent includes two portions having different degrees of expandability. The distal portion has a normal degree of expandability to support a vessel. The proximal portion has a higher degree of expandability so that it can be formed into a flange-like structure. The balloon is designed to deploy the new stent in a single operation. The balloon includes first and proximal portions having different diameters corresponding to the first and proximal portions of the stent. The distal portion is of normal diameter to deploy the distal portion of the stent in the vessel. The proximal portion is of greater diameter to form the proximal portion of the stent into the flange-like structure. The method involves deploying a conventional stent through one branch of the bifurcation, and then deploying one of the novel stent through a wall of the first stent and into the other branch. The flange-like structure on the novel stent secures the novel stent within the conventional stent. Finally, a one-piece Y-shaped stent is provided for placement in a bifurcated artery.

### BACKGROUND OF THE INVENTION

The present invention relates to cardiovascular stents and to methods of using such stents.

Cardiovascular stents are well known and are widely used in cardiovascular procedures. For example, a stent can be inserted into an artery after angioplasty to support the artery in its post-angioplasty size. Wherever used, the stents are delivered to the desired location while mounted on a balloon to facilitate movement through arteries. When the stent is in the desired location, the balloon is inflated to expand the stent and thereby deploy the stent to support the artery.

A prior art stent 10 is illustrated in FIG. 1. The stent 10 includes a plurality of generally V-shaped struts 12 interconnected in a generally tubular configuration. The V-shaped struts are generally closed when the stent is collapsed (e.g. on a balloon), and the V-shaped struts are generally opened when the stent deployed. The spacing, thickness, and strength of the struts can be varied for different applications.

A first exemplary deployment of the stent 10 is illustrated in FIG. 2 in which a first or distal portion 14 of the stent is located within an ostial vessel 40 and a second or proximal portion 16 is located within the aorta 50.

A second exemplary deployment of the stent 10 is illustrated in FIG. 3 in which the stent 10 is deployed in an ostial branch 40 extending from a primary vessel 50. In this deployment, the end 18 of the stent 10 is aligned with the wall of the primary vessel 50. This placement is particularly important if the lesion in the ostial vessel is close to the primary vessel 50. If the stent 10 is deployed too far into the ostial vessel, the lesion may not be properly supported, possibly leading to complications. On the other hand, if the stent 10 extends into the primary vessel 50, the stent can interfere with other or further intervention in that region. Consequently, the accurate placement and deployment of the stents is critical. However, even when properly deployed, a single stent is incapable of supporting all portions of a bifurcation or Y.

### SUMMARY OF THE INVENTION

The aforementioned problems are overcome in a first embodiment in which an cardiovascular stent is provided with two different radial expansion or distortion capabilities along its length. More specifically, the stent includes a first or distal portion capable of generally conventional expansion to support the artery in which the distal portion is located. The stent includes a second or proximal portion that remains outside of the ostial vessel. The proximal portion is capable of enhanced expansion, for example, up to a degree generally perpendicular to the axis of the stent to form a flange against the wall of the primary vessel.

In a second embodiment of the invention, a novel ostial balloon is provided for deploying the new stent. The balloon includes two portions having different diameters and shapes when the balloon inflated. Specifically, the balloon includes a distal portion that expands to a conventional diameter to deploy the distal portion of the stent within the ostial vessel. The balloon includes a proximal portion that expands to a substantially greater diameter to force the proximal longitudinal portion of the stent into its flange-like configuration.

In a third embodiment of the invention, the new stent of the first embodiment is used in conjunction with a conventional stent to provide full support through a bifurcation. The process includes the steps of (1) deploying a conventional stent through one branch of the bifurcation, (2) inserting the new stent through the wall of the primary stent and into the other branch of the bifurcation, (3) deploying the new stent so that the distal portion of the stent supports the other branch and the proximal portion of the stent forms a flange against the interior wall of the conventional stent.

In a fourth embodiment of the invention, a one-piece unitary stent includes an inlet portion and two outlet portions. Accordingly, the stent may be deployed in a bifurcation to fully support all three vessels meeting in the bifurcation.

The novel stents, balloon, and method have several advantages. First, the stents are more securely held in position and therefore are less subject to movement or other complications following deployment. Second, the stents and method are capable of more fully supporting plaques that are located at and through branches and bifurcations. Third, the stents and methods result in deployment that is more accurate, simple, and effective.

These and other objects, advantages, and features of the invention will be more fully understood and appreciated by reference to the description of the preferred embodiments and the drawings.

### BRIEF DESCRIPTION OF THE DRAWINGS

FIG. 1 is a side view of a prior art stent in the deployed or expanded position;

FIG. 2 is a side view of the prior art stent deployed in an ostial vessel extending from the aorta;

FIG. 3 is a side view of the prior art stent deployed in an ostial side branch vessel;

FIG. 4A is a perspective view of the stent of the present invention;

FIG. 4B is a side view of one group of struts;

FIG. 4C is an enlarged view showing the strut structure of the stent;

FIG. 5 is a side view of the stent deployed in an ostial vessel;

FIG. 6 is an end view of the deployed stent taken along line 6 in FIG. 5;

FIG. 7 is a side view of the stent deployed in an ostial branch;

FIG. 8 is an end view of the deployed stent taken along line 8 in FIG. 7;

FIG. 9 is a side view of a prior art balloon when inflated;

FIG. 10 is a side view of the ostial balloon of the present invention when fully inflated;

FIG. 11 is a side view of the stent mounted on a deflated balloon on a guide wire;

FIG. 12A is a side view of the balloon partially inflated to begin deploying the stent;

FIG. 12B is a side view of the balloon fully inflated to complete deploying the stent;

FIG. 13 is an illustration of a bifurcation with plaques;

FIG. 14 shows conventional angioplasty balloons within the bifurcation;

FIG. 15 shows a stent extending through the bifurcation and into one of the two branches;

FIG. 16 shows an inflated balloon forming an opening in the wall of the stent illustrated in FIG. 15;

FIG. 17 is a sectional view taking along line 17-17 in FIG. 16;

FIG. 18 is a sectional view similar to FIG. 17 showing the balloon deflated;

FIG. 19 shows the ostial stent extending through the opening in the first stent;

FIG. 20 shows the ostial balloon inflated to deploy the ostial stent;

FIG. 21 shows the post-procedure bifurcation supported by both the first stent and the ostial stent;

FIG. 22 is a end view of the ostial stent taken along line 22 in FIG. 21;

FIG. 23 shows the bifurcated stent of the present invention mounted on a pair of guide wires;

FIG. 24 shows the bifurcated stent on the guide wires that extend into the two branches;

FIG. 25 shows the bifurcated stent separated over the two guide wires extending into the branches;

FIG. 26 shows the bifurcated stent immediately prior to deployment;

FIG. 27 shows the bifurcated stent fully deployed; and

FIG. 28 is a schematic illustration of the aorta and primary arteries showing the locations where the stents of the present invention might be deployed.

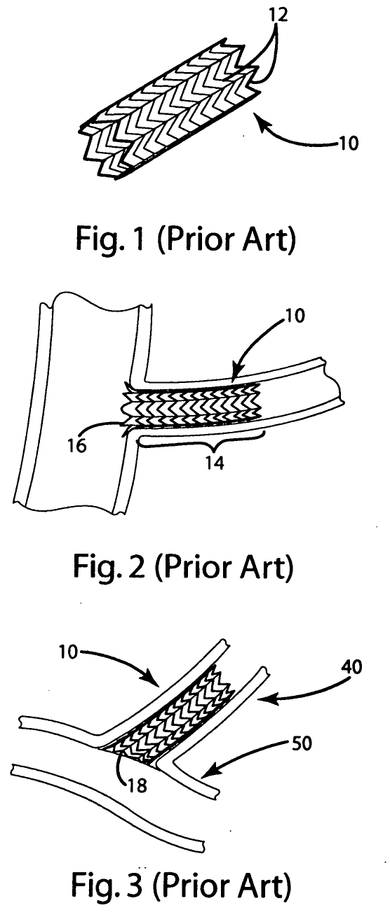

### DESCRIPTION OF THE PREFERRED EMBODIMENTS

I. Ostial Stent

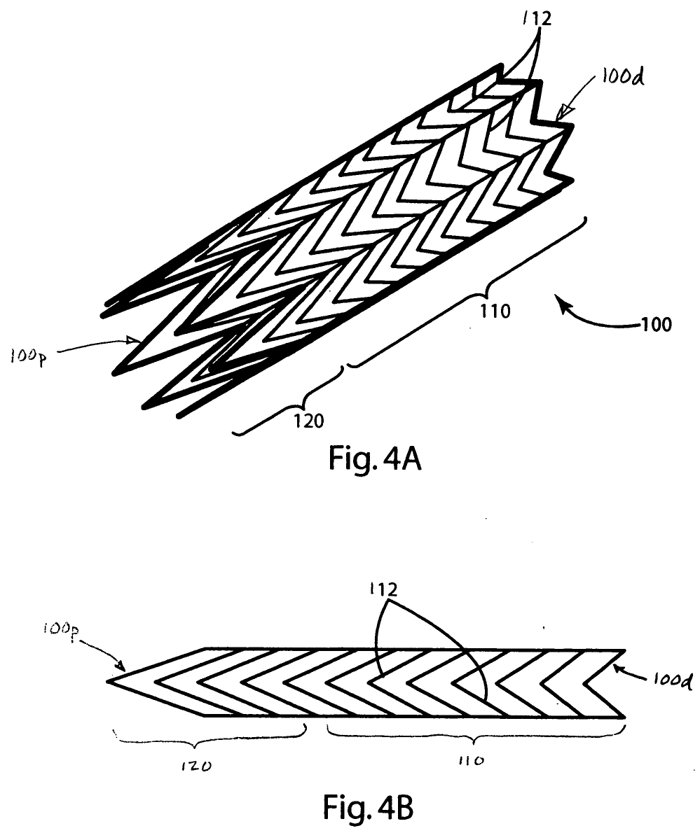

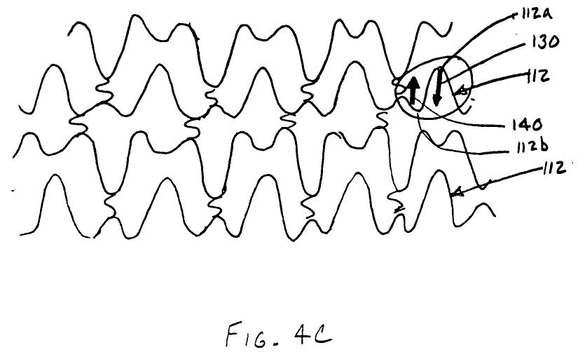

An ostial stent constructed in accordance with a preferred embodiment of the invention is illustrated in FIGS. 4-8 and generally designated 100. The stent includes a generally distal portion 110 and a generally proximal portion 120.

The struts 112 in the stent 100 are uniformly spaced along the length of the stent 100. In other words, the distance between any two struts 112 is equal. However, the lengths of the struts 112 vary along the length of the stent 100. The struts are shortest at the extreme distal end 100d, and the struts are longest at the extreme proximal end 100p. In the preferred embodiment, the length of each strut is longer than the strut on one side and shorter than the strut on the other side, so that the length of the struts 112 increases from the extreme distal end 100d to the extreme proximal end 100p.

Each of the struts is V-shaped. Because the struts vary in length, the Vs form different angles depending on the length of the strut. When the stent 100 is collapsed (as illustrated in FIG. 4A), the relatively short struts in the distal portion 110 form relatively large angles, and the relatively long struts in the proximal portion 120 form relatively small angles.

The two portions 110 and 120 therefore are capable of different radial expansions or distortions. The distal portion 110 is expandable to a degree in the range of prior art stents. Consequently, the distal portion 110 is suited for supporting a vessel. The proximal portion 120 is expandable to a far greater degree than that of the distal portion 110. Specifically, the proximal portion 120 may be expanded until it is generally perpendicular to the axis of the stent thereby forming a flange on the distal portion 110.

FIG. 4C is an enlarged view showing more specifically the presently envisioned structure for the struts 112. Arrows 130 and 140 show the direction of movement of the strut portions 112a and 112b respectively as the stent expands. The two directions are opposite one another. The specific FIG. 4C structure enables the struts 112 to be uniformly spaced (i.e. the distance between adjacent struts is constant).

The disclosed stent 100 is but one example for constructing a strut having different degrees of expandability or distortion along its length. The length of the struts can vary in a fashion other than as described. Further, other techniques for providing different portions or areas of expansion or distortion will be known to those skilled in the art.

A circumferential marker (not illustrated) is located on the outer wall of the stent in conventional fashion to assist the physician in properly locating the stent during the procedure.

The stent 100 provides significant flexibility, accommodates angled vessels without losing the integrity of the stent, and enables deployment of the stent in vessels whose diameters vary along the location of the stent.

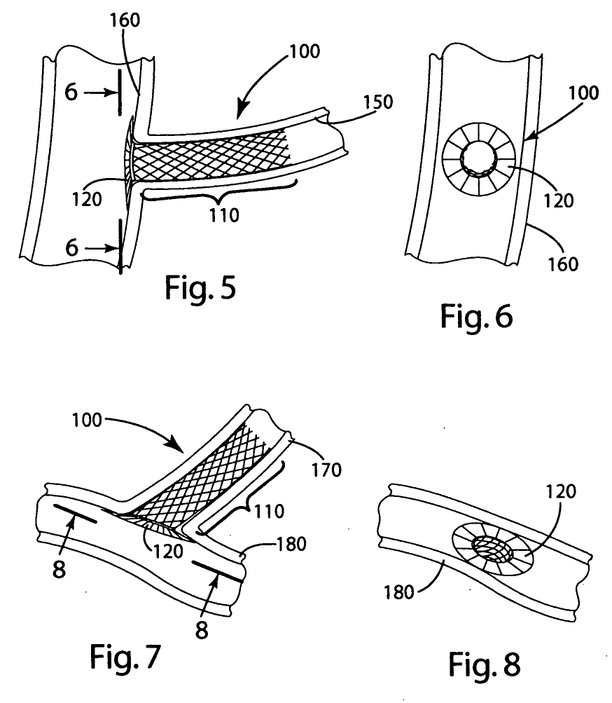

FIGS. 5-6 illustrate the deployment of the stent 100 within an ostial branch 150 extending from a primary vessel 160. The distal portion 110 of the stent is located within the branch 150, and the proximal portion 120 is flared outwardly to form a flange against the interior wall of the primary vessel 160. When so deployed, the distal portion 110 supports the vessel 150. The flange 120 assists in maintaining the stent 100 in proper position subsequent to the catheterization procedure.

FIGS. 7-8 show the stent 100 deployed in an ostial branch 170 extending from a primary vessel 180. The distal portion 110 of the stent 100 is located within the ostial branch 170 to support the branch. The proximal portion 120 is flared outwardly to form a flange against the interior wall of the ostial vessel 180. The marker is aligned with the wall of the primary vessel 180.

The ostial stent can be mounted on a conventional balloon for deployment in “straight” vessels. The struts for straight stents would have an angulation in the approximate range of 45 degrees to 55 degrees in comparison to the approximate range of 30 degrees to 60 degrees in the above described ostial stent. The greater angulation provides appropriate support for proximal vessel walls.

II. Ostial Balloon

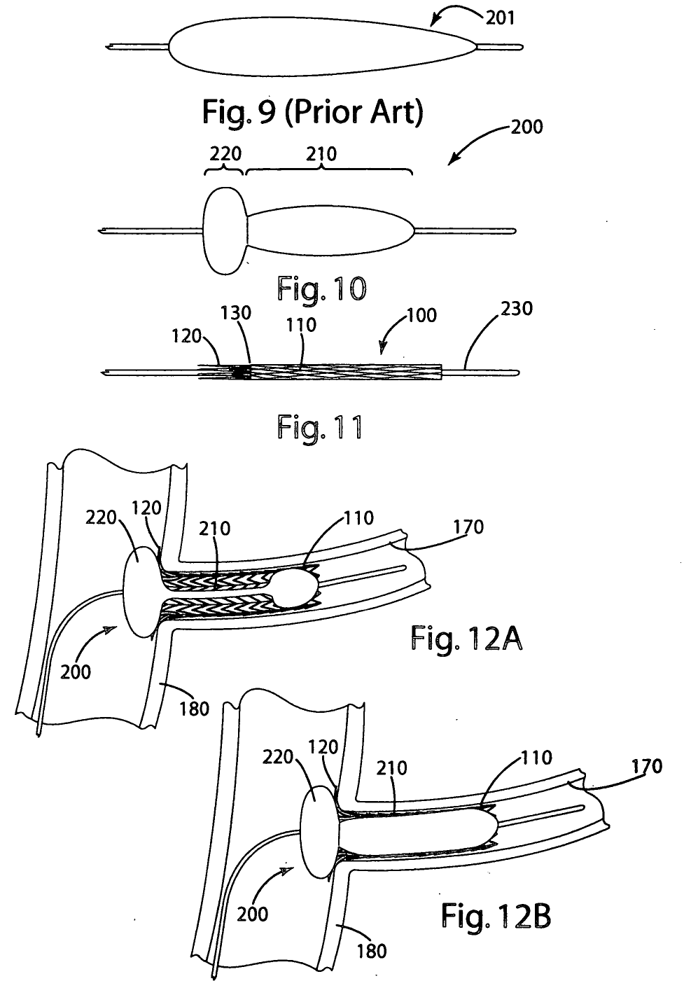

A prior art stent balloon 200 is illustrated in FIG. 9. The prior art balloon is ovoid or cigar-shaped when inflated as illustrated in FIG. 9. As is well known in the art, the stent is mounted over the deflated balloon before the stent/balloon combination is delivered to the desired location. When the stent is properly positioned, the balloon is inflated to expand or deploy the stent into its operative configuration. The prior art balloon 200 is incapable of fully deploying the stent 100. Specifically, the prior art balloon 200 cannot expand the proximal portion 120 of the stent 100 into its flange-shaped configuration.

An ostial balloon constructed in accordance with a preferred embodiment of the invention is illustrated in FIGS. 10-12 and generally designated 200. The balloon includes a first longitudinal portion 210 and a second longitudinal portion 220. The first longitudinal portion when fully inflated (FIG. 12B) is essentially ovoid as in the prior art. The second portion 220 is bulbous when fully inflated, and has a diameter substantially greater than that of the distal portion 210. The first and second portions 210 and 220 are integrally connected to one another. Consequently, inflation of the balloon 200 results in inflation of both portions 210 and 220. Methods and techniques for fabricating the balloon will be known to those skilled in the art.

The stent 100 is shown in FIG. 11 in its collapsed condition mounted over a balloon (not visible) on a guide wire 230. As is well known to those skilled in the art, the guide wire 230 is used to guide the stent 100 to the deployment location.

FIGS. 12A and B illustrate the ostial balloon 200 at two different stages of inflation to deploy the stent 100. FIG. 12A shows the first balloon portion partially inflated, and FIG. 12B shows the first balloon portion fully inflated. FIG. 12A illustrates the initial inflation of the balloon in which the distal end of the distal portion is inflated and in which the proximal portion is inflated. As inflation continues, the balloon inflates toward the center from the opposite ends. When full inflated (FIG. 12B), the distal portion 210 of the balloon deploys the distal portion 110 of the stent 100. The described inflation sequence traps all plaques within the stent, preventing a distal embolization. Similarly, the proximal portion 220 of the balloon deploys the proximal portion 120 of the stent 100. Because the distal portion 210 is substantially similar in size and shape to the prior art stent 201 (see FIG. 9), this portion of the balloon properly deploys the supportive portion 110 of the stent 100 within the ostial branch 170. Because the proximal portion 220 has a diameter substantially greater than that of the distal portion 210, and further because of its bulbous shape as it inflates, the proximal portion 220 forms the proximal portion 120 of the stent into a flange against the wall of the main branch 180.

III. Procedure Using Both Conventional Stent and the New Ostial Stent

The ostial stent 100 can be used in conjunction with a conventional stent 10 (or another ostial stent) to fully support a bifurcation or branch in which an incoming vessel and two outgoing vessels meet in a Y. Further, the procedure results in a combination device that fully support all areas within and through the bifurcation.

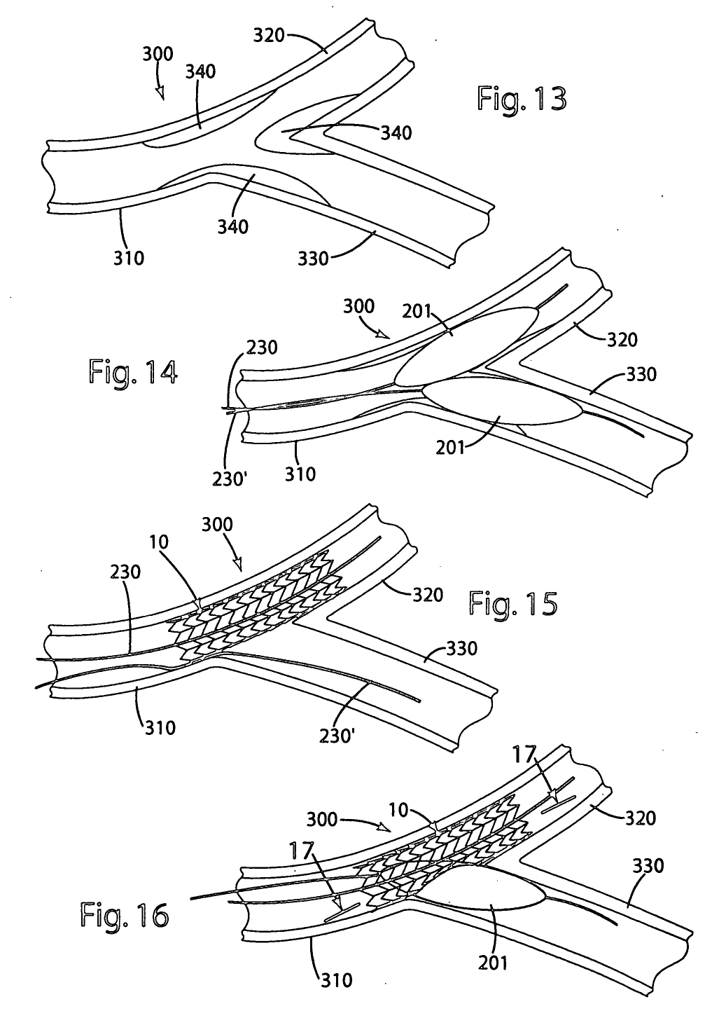

A bifurcation 300 is illustrated in FIG. 13. The bifurcation includes an incoming or main branch 310 and two outgoing or extending branches 320 and 330. As disclosed, plaques 340 might exist in any or all of the variety of areas illustrated.

The first step in treating the plaques is conventional kissing angioplasty as illustrated in FIG. 4. A pair of conventional balloons 201 are inserted using guide wires 230 and 230′ in conventional fashion. The balloons are inflated to perform the angioplasty and to restore the branches 310, 320 and 330 to their original diameters. The angioplasty balloons are then deflated and withdrawn.

A conventional stent 10 (or an ostial stent as described in this application) is subsequently inserted and deployed as illustrated in FIG. 15. The stent 10 extends through the bifurcation from the incoming branch 110 to the outgoing branch 320. When so deployed, the second branch 330 is closed by the stent 10.

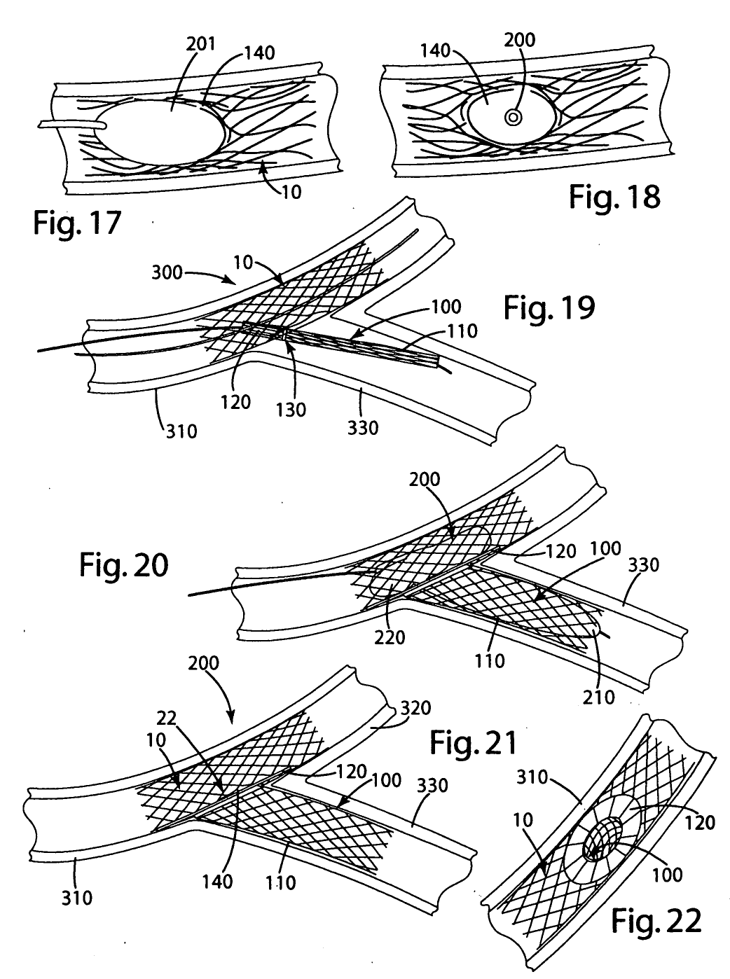

The guide wire 230′ is then withdrawn from the branch 330 and is advanced into the stent 110, through the wall (between the struts 112) of the stent 10, and into the branch 330. A conventional balloon 201 (or an ostial balloon as described in this application) is then positioned on the wire 230′ and through the wall of the stent 10. If a new ostial stent is used, rather than a conventional stent, the balloon can be more easily inserted between the struts. The balloon 201 is then inflated or expanded. The procedure at this point is illustrated in FIG. 16-17. The inflated balloon creates an opening 140 through the wall of the stent 10 by moving the struts. The diameter of the opening 140 is approximately the same as the diameter of the branch 330.

The balloon 200 is then deflated as illustrated in FIG. 18. The opening 140 remains the same size as created by the balloon. The balloon then is withdrawn from the conventional stent 10.

The next step is to position an ostial stent 100 in the opening 140 through the wall of the conventional stent 10. The result of this step is illustrated in FIG. 19. The distal portion 110 of the stent is located within the branch 330 and the proximal portion 120 of the stent is located within the conventional stent 10.

The balloon 200 on which the stent 100 is mounted is then inflated as illustrated in FIG. 20. When the balloon is fully inflated, the distal portion 110 of the stent is expanded or deployed to support the branch 330, and the proximal portion 220 of the balloon forces the proximal portion 120 of the stent into a flange-like configuration against the wall of the conventional stent 10. The balloon 200 and both wires 230 and 230′ are then withdrawn so that the final result is as illustrated in FIGS. 21 and 22.

The resulting two-stent combination fully supports all areas in and through the entire bifurcation. The method therefore provides previously unavailable treatment in a relatively simple but effective procedure.

IV. One-Piece Bifurcated Stent

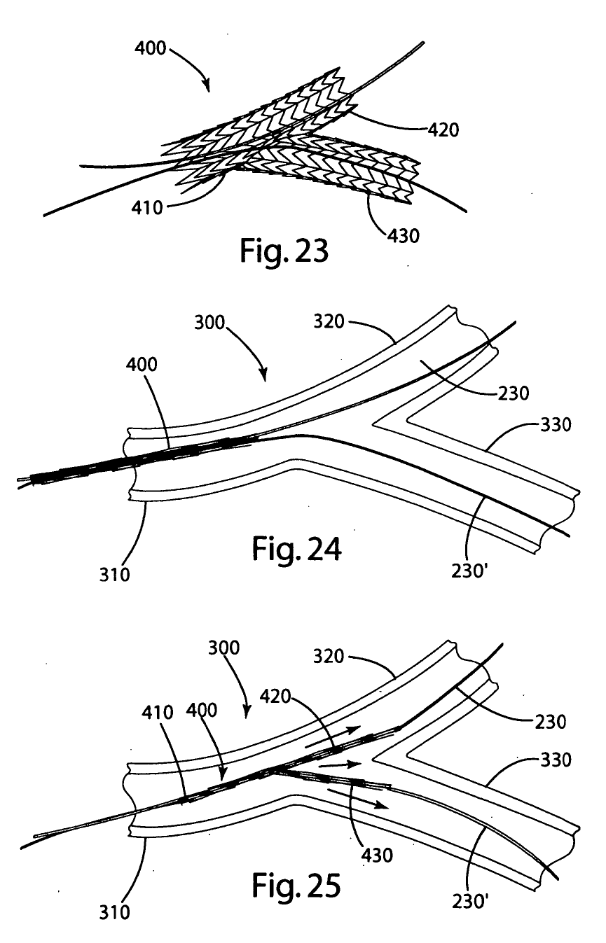

A one-piece, unitary stent for deployment in a bifurcation is illustrated in FIGS. 23-27 and generally designated 400. The stent includes an inlet portion 410 and two outlet (or ostial) portions 420 and 430. The three portions form a Y. All of the portions 410, 420 and 430 are part of a single integrated whole. Methods and techniques fabricating the stent 400 will be apparent to those skilled in the art.

The bifurcated stent 400 is deployed in a bifurcation 300 as illustrated in FIGS. 24-27. FIG. 24 shows the stent 400 mounted on the guide wires 230 and 230′. The guide wire 230 extends through portions 410 and 420, and the guide wire 230′ extends through portions 410 and 430.

FIG. 25 shows the two portions 420 and 430 following the guide wires 230 and 230′ respectively into the branches 320 and 330 respectively.

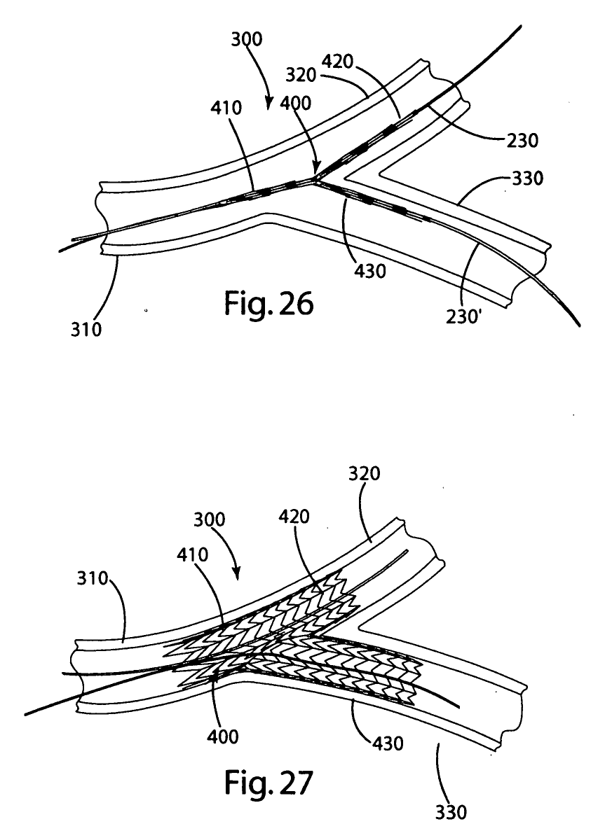

FIG. 26 shows the position of the stent 400 just prior to deployment. The three stent portions 410, 420, and 430 are positioned within the three branches 310, 320, and 330 respectively.

FIG. 27 shows the stent 400 following deployment. The three stent portions 410, 420, and 430 support the respective bifurcation portions 310, 320 and 330.

The balloon (not shown) for deploying the stent 400 is Y-shaped. Its construction and fabrication will be apparent to those skilled in the art.

IV. Conclusion

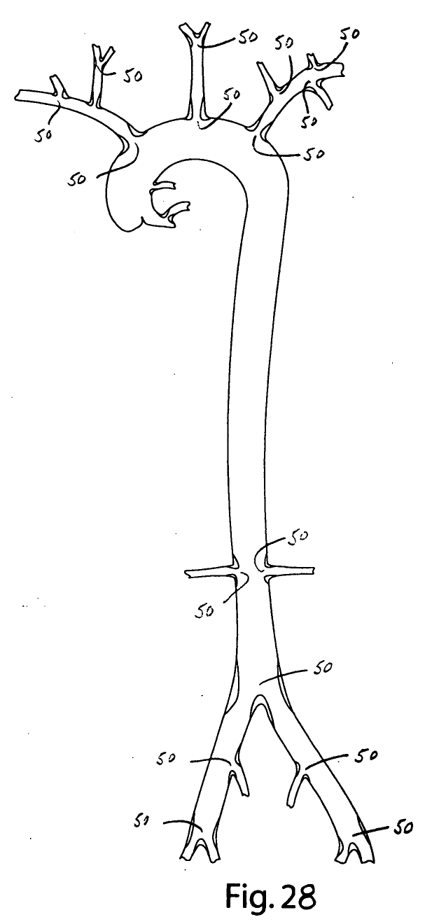

FIG. 28 is a schematic illustration of the aorta and primary arteries showing some of the possible locations 50 in which the stents of the present invention might be deployed. As can be seen, the possible locations are widespread and varying.

The above described stents and procedures enhance and expand cardiovascular procedures. The stents and procedures are highly effective and enable a variety of new areas, such as bifurcations, to be stented. The stents are less subject to movement and other subsequent complications.

The above descriptions are those of current embodiments of the invention. Various alterations and changes can be made without departing from the spirit and broader aspects of the invention, which are to be interpreted in accordance with the principles of patent law including the Doctrine of Equivalents.

1. An cardiovascular stent comprising:
a first portion having at least a first degree of expandability; and
a second portion having at least a second degree of expandability greater than the first degree of expandability, said second longitudinal portion terminating in one end of the stent, whereby said one end can be expanded beyond said first longitudinal portion.

2. A cardiovascular stent as defined in claim 1 wherein said stent comprises struts, the length of the struts in the first portion differing from the length of the struts in the second portion.

3. A method of treating a secondary cardiovascular vessel extending from a primary cardiovascular vessel, said method comprising the steps of:
providing a stent having distal and proximal portions, the proximal portion being more expandable than the distal portion;
positioning the stent so that the distal portion of the stent is located in the secondary vessel and the proximal portion of the stent is located in the primary vessel;
expanding the distal portion of the stent to support the secondary vessel; and
expanding the proximal portion of the stent to form a flange engaging the primary vessel.

4. A method as defined in claim 3 wherein said providing step includes providing the stent with struts of varying length along the length of the stent.

5. A stent balloon comprising:
a distal portion having a first diameter when inflated; and
a proximal portion having a second diameter when inflated, the second diameter being greater than the first diameter, whereby said balloon is capable of expanding a stent in which it is positioned to two different extents corresponding to the first and second diameters.

6. A stent balloon as defined in claim 5 wherein:
said distal portion is ovoid shaped; and
said proximal portion is bulbously shaped.

7. A method of treating a cardiovascular bifurcation including an inlet portion and two outlet portions meeting in a junction, said method comprising the steps of:
inserting a first stent in the inlet portion and one of the outlet portions on either side of the junction;
expanding the first stent to support the inlet portion and the one outlet portion;
inserting a second stent through a wall of the first stent so that (1) a distal portion of the second stent is located in the other outlet portion, and (2) a proximal portion of the second stent is located within the first stent;
expanding the distal portion of the second stent to support the other outlet portion; and
expanding the proximal portion of the second stent so that the proximal portion engages the inner wall of the first stent in a flange-like arrangement.

8. A method as defined in claim 7 wherein said inserting step includes inserting a stent having struts of varying length along the length of the stent.

9. An arterial stent comprising:
an inlet portion;
first and second outlet portions in fluid communication with said inlet portion; and
said inlet portion and said first and second outlet portions fabricated as a single unitary piece, whereby said stent may be deployed in a cardiovascular bifurcation as a single, unitary piece supporting the inlet and both outlets of the bifurcation.
